(19) United States
(12) Patent Application Publication
(10) Pub. No.: US 2008/0050411 A1
(43) Pub. Date: Feb. 28, 2008

(54) TEXAS GAME DRAW

(76) Inventor: Jonathan David Rudd, Bryan, TX (US)

Correspondence Address: JON DAVID RUDO, 2920 MONTANA, BRYAN, TX, 77803, US

(21) Appl. No.: 11/365,312

(22) Filed: Aug. 28, 2006

(51) Int. Cl. A61K 36/899

(52) U.S. Cl. 424/410; 424/750

(57) ABSTRACT

The present invention is a game attractant comprised of a mixture of rice bran, wheat bran and dried molasses to attract the attention of ungulates and avian fauna. By the incorporation of these three ingredients, the nutritional and vitamin value is elevated above the natural diet of the fauna. Therefore once expelled in pyramid form upon the intended-sight, the animals are attracted to the richness of this mix.

The present invention is for a wild game attractant comprised of rice bran, wheat bran and dried molasses.

The first ingredient in this invention is rice bran, which is a by-product of rice milling. Rice bran is source of Vitamin B1, Thiamine, starch, protein, and fiber. The second ingredient is wheat bran, a by-product of wheat milling. Wheat bran is a source of fiber, fat, and protein. The third and final ingredient is dried molasses which is a by-product of the sugar industry. Dried molasses is rich in a multitude of different sugars which provides a source of energy. Also this ingredient acts as a binding agent to adhere all reagents in the invention. Both rice and wheat bran are extremely fine and require the addition of the dried molasses to provide for a consistent mixture of the invention.

By mixing 16 parts rice bran, eight parts wheat bran and four part dried molasses into a commercial mixer for 20 minutes, (one part equals one pound). The resultant is a rough tan powder. This powder is then allocated into plastic holding containers. A foam lined lid is utilized to seal the plastic holding container. This closure provides an air tight method for storage of the invention.

Once the container is brought to the intended sight, the lid is unscrewed and the product is deposited onto the designated area in a pyramid shape mound. This mound is necessary for the ungulates and avian fauna to consume the product because of the fine particles that compose the product.

Once the product is consumed, a small portion of the product remains in the soil. This small portion of product provides a substantial amount of scent that continues to draw the animals to the site of deposition causing them to repeatedly return to the same area. Appling the product as necessary results in the reoccurrence of the fauna to the same sight.

While the invention has been described as above, some modifications and variations of the invention may be constructed without departing from the scope of the invention as defined in the following claims.

I claim:

1. A method of attracting wild game comprised of rice bran, wheat bran and dried molasses expelled onto an intended sight on the forest floor or natural animal-pathway where wild animals are prone to migrate from feeding to bedding.

2. The method of claim 1 utilizes the organic properties of the rice bran as an attractant because of the high fat properties and high vitamin E.

3. The method of claim 1 utilizes the organic properties of the wheat bran as an attractant because of the high content of crude protein and valuable starch.

4. The method of claim 1 utilizes the organic properties of the dried molasses as an attractant because it stimulates the appetite of the animal, gives them energy and binds claim number 2 and claim number 1 together.

5. The method of claim 1 attracts members of the order artiodactyla, perissodactyla and from the class aves for viewing.
